(19) United States
(12) Patent Application Publication
(10) Pub. No.: US 2022/0183614 A1
(43) Pub. Date: Jun. 16, 2022

(54) METHOD OF PREDICTING PERSONALIZED POLLEN ALLERGY USING POLLEN CALENDAR AND PERSONAL ALLERGIC SYMPTOM DIARY AND SERVER PERFORMING THE SAME

(71) Applicant: National Institute of Meteorological Sciences, Seogwipo-si (KR)

(72) Inventors: Kyu Rang Kim, Gangneung-si (KR); Mae Ja Han, Gangneung-si (KR); Ju Young Shin, Gangneung-si (KR); Seung Bum Kim, Gangneung-si (KR); Jae Won Oh, Guri-si (KR)

(73) Assignee: National Institute of Meteorological Sciences, Seogwipo-si (KR)

(21) Appl. No.: 17/643,459

(22) Filed: Dec. 9, 2021

(30) Foreign Application Priority Data
Dec. 16, 2020 (KR) 10-2020-0176222

(51) Int. Cl. A61B 5/00; G16H 50/30

(52) U.S. Cl. CPC A61B 5/411; G16H 50/30

(57) ABSTRACT

A method of predicting a personalized pollen allergy includes generating a personal allergic symptom diary by recording a daily allergic symptom and daily drug taking information of a user, calculating a daily symptom index using a pollen calendar of a region corresponding to a location of the user and the daily allergic symptom, extracting allergy generation risk grades for each pollen generation species and allergy-sensitive tree species of the user by using the pollen generation species and a pollen generation grade extracted from the pollen calendar, and the daily symptom index, and generating a personalized pollen calendar based on the extracted information, and generating a personalized risk forecast for each city and county for the user by applying the allergy generation risk grades for each pollen generation species and the allergy-sensitive tree species to a Metrological Administration pollen forecast.

### CROSS-REFERENCE TO RELATED APPLICATION

This application claims priority to and the benefit of Korean Patent Application No. 2020-0176222, filed on Dec. 16, 2020, in the Korean Intellectual Property Office, the disclosure of which is incorporated herein by reference in its entirety.

### BACKGROUND

### 1. Field

The present disclosure relates to a method of predicting a personalized pollen allergy, which uses a pollen calendar and an allergy patient symptom diary, and a server performing the same. More particularly, the present disclosure relates to a method of predicting personalized pollen allergy, which is capable of providing a personalized pollen allergy prediction service by combining a pollen calendar and a patient's pollen allergic symptom, and a server performing the same.

### 2. Description of Related Art

Pollen is one of the causative agents of allergic rhinitis, conjunctivitis, or the like, and allergic symptoms caused by pollen are collectively called a pollen allergy.

The pollen allergy is mainly caused by anemophilous flowers that scatter a good deal of pollen in the air. The amount of pollen scattered in the air is greatly affected by a density of vegetation and weather conditions.

An average concentration of pollen may be calculated based on data obtained by observing a concentration of pollen in the air on a daily basis for a long period of time, and a pollen calendar based on the average concentration of pollen may be obtained. The pollen calendar expresses the average concentration of pollen by region, tree type, and day.

Meanwhile, pollen allergy symptoms of users are affected by a type and concentration of pollen. However, there is a problem in that the information on the conventional pollen calendar does not provide a combination of information on the concentration of pollen and information on allergic symptoms for each user.

### SUMMARY

The present disclosure is directed to providing a method of predicting a personalized pollen allergy, which is capable of providing a personalized pollen allergy prediction service by combining a pollen calendar with patients' pollen allergic symptoms, and a server performing the same.

Problems to be solved by the present disclosure are not limited to the above-mentioned aspects. That is, other aspects that are not described may be obviously understood by those skilled in the art from the following specification.

According to an aspect of the present disclosure, there is provided a method of predicting a personalized pollen allergy, comprising: generating a personal allergic symptom diary by recording a daily allergic symptom and daily drug taking information of a user; calculating a daily symptom index using a pollen calendar of a region corresponding to a location of the user and the daily allergic symptom; extracting allergy generation risk grades for each pollen generation species and allergy-sensitive tree species of the user by using the pollen generation species and a pollen generation grade extracted from the pollen calendar, and the daily symptom index, and generating a personalized pollen calendar based on the extracted information; and generating a personalized risk forecast for each city and county for the user by applying the allergy generation risk grades for each pollen generation species and the allergy-sensitive tree species to a Metrological Administration pollen forecast.

According to another aspect of the present disclosure, there is provided a personalized pollen allergy prediction server, including: an allergic symptom diary generation unit configured to generate a personal allergic symptom diary by recording a daily allergic symptom and daily drug taking information of a user; a symptom index calculation unit configured to calculate a daily symptom index using a pollen calendar of a region corresponding to a location of the user and the daily allergic symptom; a personalized pollen calendar generation unit configured to extract allergy generation risk grades for each pollen generation species and allergy-sensitive tree species of the user by using the pollen generation species and a pollen generation grade extracted from the pollen calendar, and the daily symptom index, and generate a personalized pollen calendar based on the extracted information; and a risk forecast generation unit configured to generate a personalized risk forecast for each city and county for the user by applying the allergy generation risk grades for each pollen generation species and the allergy-sensitive tree species to a Metrological Administration pollen forecast.

### BRIEF DESCRIPTION OF THE DRAWINGS

The above and other aspects, features and advantages of certain embodiments of the present disclosure will become more apparent from the following description taken in conjunction with the accompanying drawings, in which:

FIG. 1 is a network configuration diagram illustrating a system for providing a personalized pollen allergy prediction service according to an embodiment of the present disclosure;

FIG. 2 is a block diagram for describing a configuration of a personalized pollen allergy prediction server according to an embodiment of the present disclosure;

FIG. 3 is a flowchart for describing a method of predicting a personalized pollen allergy according to an embodiment of the present disclosure;

FIG. 4 is an exemplary diagram for describing the execution process of FIG. 3 and is a diagram illustrating a pollen calendar in Seoul, Korea;

FIG. 5 is an exemplary diagram for describing the execution process of FIG. 3 and is a diagram illustrating a Korea Metrological Administration pollen forecast screen provided by the server illustrated in FIG. 2; and

FIG. 6 is a hardware configuration diagram of a computing device capable of implementing a server in a system for providing a personalized pollen allergy prediction service according to some embodiments of the present disclosure.

### DETAILED DESCRIPTION

Throughout the specification, like reference numerals denote like elements. The present specification does not describe all elements of embodiments, and general content in the technical field to which the present disclosure pertains or content that overlaps between embodiments will be omitted.

The terms “unit, “module”, “member”, or block” used in the specification may be implemented in software or hardware, and according to embodiments, a plurality of “units, modules, members, blocks” may be implemented as one component, or one “unit, module, member, block” can also include a plurality of components.

Throughout the specification, “connecting” any part to another part includes not only direct connection but also indirect connection, and the indirect connection includes connection through a wireless communication network.

In addition, unless explicitly described to the contrary, “including” any component will be understood to imply the inclusion of other components rather than the exclusion of other components.

Throughout the specification, when any member is referred to as being positioned “on” another member, it includes not only a case in which any member and another member are in contact with each other, but also a case in which the other member is interposed between any member and another member.

The terms “first,” “second,” and the like are used to distinguish one element from another element, and the elements are not defined by the above-described terms.

Singular forms are intended to include plural forms unless the context clearly makes an exception.

In each step, an identification symbol is used for convenience of description, and the identification symbol does not describe the order of each step, and each step may be performed differently from the specified order unless the specific order is clearly stated in the context.

Embodiments of the present disclosure will be described with reference to the accompanying drawings.

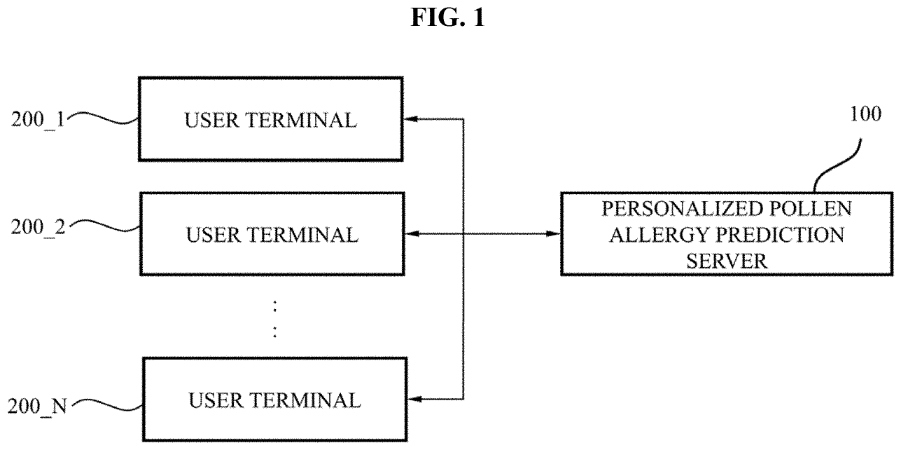

FIG. 1 is a network configuration diagram for describing a system for providing a personalized pollen allergy prediction service according to an embodiment of the present disclosure.

Referring to FIG. 1, the system for providing a personalized pollen allergy prediction service includes a personalized pollen allergy prediction server 100 and user terminals 200_1 to 200_N.

The personalized pollen allergy prediction server 100 (hereinafter referred to as “server”) predicts personalized pollen allergy based on daily pollen allergic symptoms and a pollen calendar and provides the predicted personalized pollen allergy to the user terminals 200_1 to 200_N.

To this end, the server 100 generates a personal allergic symptom diary by recording daily pollen allergic symptoms (hereinafter, referred to as “allergic symptoms”).

That is, the server 100 may generate a personal allergic symptom diary using a pollen calendar, in which pollen generation information by day is displayed, a daily allergic symptom, and daily drug taking information.

In this case, the allergic symptom includes primary symptoms and secondary symptoms. The primary symptom may include one or more of sneezing, clear nasal discharge, stuffy nose, nasal itching, difficulty in smelling, and the like. The secondary symptom includes headache, mouth breathing, post nasal drip syndrome, coughing while sleeping, sleep disorder, and the like.

After generating the personal allergic symptom diary, the server 100 may obtain daily symptom indexes by counting each of the daily allergic symptoms. According to an embodiment, a counting method may vary depending on whether a user takes a drug.

In one embodiment, when a user does not take a drug, the server 100 determines a first weight depending on whether the recorded allergic symptom is the primary symptom or the secondary symptom. Then, the server 100 calculates symptom indexes for each allergic symptom by adding the first weight to each of the number of allergic symptoms and the duration of each allergic symptom. Then, a first final symptom index may be calculated by summing the symptom indexes for each allergic symptom. The server 100 may calculate the first final symptom index by day by repeating the above-described process for the daily allergic symptoms.

In another embodiment, when a user is taking a drug, the server 100 generates a symptom alleviation time for each allergic symptom by comparing an allergic symptom (for example, first duration, etc.) before taking the drug and an allergic symptom (for example, second duration, etc.) according to alleviation information of the drug after taking the drug. The symptom indexes for each allergic symptom are calculated by adding a second weight to each of the number of allergic symptoms and the symptom alleviation time for each allergic symptom. Thereafter, a second final symptom index may be calculated by summing the symptom indexes for each allergic symptom. The server 100 may calculate the second final symptom index by day by repeating the above-described process for the daily allergic symptoms.

As described above, the second weight is a weight reflected in the symptom alleviation time for each allergic symptom when the user takes the drug. The second weight may be set higher as the number of times the user takes the drug increases. This is because the symptoms may be alleviated more as the number of times the user takes the drug increases. On the other hand, the second weight may be set lower as the number of times the user takes the drug decreases. This is because the symptoms may become more severe as the number of times the user takes the drug decreases.

Then, the server 100 extracts pollen generation information by date from a pollen calendar of a region corresponding to a location of a user. The pollen generation information may include a pollen generation species (tree type) and a pollen generation grade. The pollen generation grade may be divided into, for example, “low,” “moderate,” “high,” and “very high.” Then, the server 100 extracts allergy generation risk grades for each pollen generation species and allergy-sensitive tree species based on the pollen generation species, the pollen generation grade, and the final symptom index calculated in advance. Hereinafter, embodiments related to the extraction of the allergy generation risk grades for each pollen generation species and the extraction of the allergy-sensitive tree species will be described.

In one embodiment, the server 100 extracts the pollen generation species by date from the pollen calendar of the region corresponding to the location of the user and extracts the final symptom index recorded on the same date as the date of the pollen calendar among the dates of the personal allergic symptom diary. In this case, the extracted final symptom index may be one of the first final symptom index and the second final symptom index.

Thereafter, the server 100 displays the first final symptom indexes or the second final symptom indexes for each pollen generation species on a graph of the allergy generation risk grades for each symptom index. Then, the server 100 extracts the allergy generation risk grades for each pollen generation species according to an area in which the first final symptom index or the second final symptom index is located on the graph of the allergy generation risk grades for each symptom index.

In another embodiment, the server 100 extracts the date corresponding to the same pollen generation species and the same pollen generation grade from the pollen calendar. For example, suppose a pollen generation grade for oak species is maintained at “high” from April 20 to April 30 in the pollen calendar. In this case, the server 100 extracts dates from April 20 to April 30 in the pollen calendar. Then, the server 100 extracts the first final symptom index and/or the second final symptom index recorded on the date corresponding to the date extracted from the pollen calendar among the dates of the personal allergic symptom diary. The extracted first final symptom index and/or second final symptom index is displayed on the graph of the allergy generation risk grades for each index. Then, the server 100 extracts the allergy generation risk grades for the oak species according to the area in which the first final symptom index and/or the second final symptom index is located on the graph of the allergy generation risks for each symptom index. The server 100 repeats this process to extract the allergy generation risk grades for each pollen generation species.

In the above embodiment, when a difference between the allergy generation risk grade corresponding to the first final symptom index and the allergy generation risk grade corresponding to the second final symptom index is greater than or equal to a reference value, the server 100 determines the higher of the two allergy generation risks as the final allergy generation risk grade of the user.

As such, when the final allergy generation risk grades are extracted for each pollen generation species, the server 100 may determine a pollen generation species with a final allergy generation risk grade above a specific grade as the allergy-sensitive tree species.

Meanwhile, the server 100 compares the time when the allergy generation risk grades for each pollen generation species of each user persist to the specific grade or higher and the time when the pollen generation grades extracted from the pollen calendar persist to the specific grade or higher to determine the allergy-sensitive tree species for each user and then generate the personalized pollen calendar in which the allergic symptoms are displayed.

In addition, the server 100 may compare the time when the daily symptom indexes of the user are maintained at a certain level or more and the time when the allergy generation risk grades for each pollen generation species are maintained at a specific level to determine a personal allergy-sensitive tree species and generate a personalized pollen calendar in which allergic symptoms are displayed.

Then, the server 100 applies the allergy generation risk grade for each pollen generation species and the allergy-sensitive tree species to the Korea Metrological Administration pollen forecast to generate a personalized risk forecast for each city and county in which information on a region in which allergic symptoms may appear is spatially represented in more detail compared to the Korea Metrological Administration pollen forecast.

The user terminals 200_1 to 200_N are terminals owned by users. The user terminals 200_1 to 200_N receive and utilize the personalized risk information, that is, the personalized risk forecast for each city and county from the server 100. These user terminals 200_1 to 200_N may be implemented as a tablet personal computer (PC), a smart phone, or the like.

The user terminals 200_1 to 200_N provide the server 100 with the allergic symptom occurring in the user and the drug taking information of the user. Accordingly, the server 100 may record the allergic symptoms and the drug taking information by day to generate the personal allergic symptom diary.

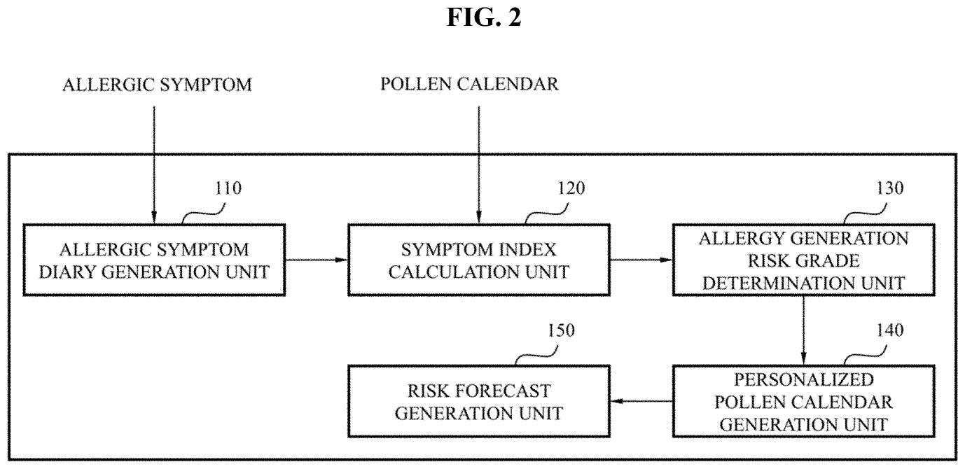

FIG. 2 is a block diagram for describing a configuration of a personalized pollen allergy prediction server according to an embodiment of the present disclosure.

Referring to FIG. 2, the server 100 includes an allergic symptom diary generation unit 110, a symptom index calculation unit 120, an allergy generation risk grade determination unit 130, a personalized pollen calendar generation unit 140, and a risk forecast generation unit 150.

The allergic symptom diary generation unit 110 stores the daily allergic symptoms and daily drug taking information to generate an allergy patient symptom diary.

The allergic symptom includes the primary symptom and the secondary symptom. The primary symptom may include one or more of sneezing, clear nasal discharge, stuffy nose, nasal itching, difficulty in smelling, and the like. The secondary symptom includes headache, mouth breathing, post nasal drip syndrome, coughing while sleeping, sleep disorder, and the like.

The symptom index calculation unit 120 calculates the symptom index using the pollen calendar of the region corresponding to the location of the user among the pollen calendars, in which the pollen generation information by date is displayed, and the personal allergic symptom diary generated by the allergic symptom diary generation unit 110.

In one embodiment, the symptom index calculation unit 120 determines the first weight depending on whether the recorded allergic symptom is a primary symptom or a secondary symptom when the user does not take the drug. Then, the server 100 calculates symptom indexes for each allergic symptom by adding the first weight to each of the number of allergic symptoms and the duration of each allergic symptom. Thereafter, a first final symptom index may be calculated by summing the symptom indexes for each allergic symptom. The symptom index calculation unit 120 may calculate the first final symptom index by day by repeating the above-described process for the daily allergic symptoms.

In another embodiment, when the user is taking the drug, the symptom index calculation unit 120 compares the allergic symptom (for example, the first duration, etc.) before taking drugs for each allergic symptom and the allergic symptom (for example, the second duration, etc.) according to the drug alleviation information after taking drugs for each allergic symptom to generate the symptom alleviation time. The symptom indexes for each allergic symptom are calculated by adding a second weight to each of the number of allergic symptoms and the symptom alleviation time for each allergic symptom. Thereafter, a second final symptom index may be calculated by summing the symptom indexes for each allergic symptom. The symptom index calculation unit 120 may calculate the second final symptom index by day by repeating the above-described process for the daily allergic symptoms.

As described above, the second weight is a weight reflected in the symptom alleviation time for each allergic symptom when the user takes the drug. The second weight may be set higher as the number of times the user takes the drug increases. This is because the symptoms may be alleviated as the number of times the user takes the drug increases. On the other hand, the second weight may be set lower as the number of times the user takes the drug decreases. This is because the symptoms may become more severe as the number of times the user takes the drug decreases.

Then, the allergy generation risk grade determination unit 130 extracts the pollen generation information by date from the pollen calendar of the region corresponding to the location of the user. The pollen generation information may include a pollen generation species and a pollen generation grade. The pollen generation grade may be divided into, for example, “low,” “moderate,” “high,” and “very high”. Then, the allergy generation risk grade determination unit 130 extracts the allergy generation risk grades for each pollen generation species and allergy-sensitive tree species by using the pollen generation species, the pollen generation grade, and the final symptom index calculated by the symptom index calculation unit 120. Hereinafter, embodiments related to the extraction of the allergy generation risk grades for each pollen generation species and the extraction of the allergy-sensitive tree species will be described.

In one embodiment, the allergy generation risk grade determination unit 130 extracts the pollen generation species by date from the pollen calendar of the region corresponding to the location of the user, and extracts the final symptom index recorded on the same date as the date of the pollen calendar among the dates of the personal allergic symptom diary. In this case, the extracted final symptom index may be one of the first final symptom index and the second final symptom index.

Thereafter, the allergy generation risk grade determination unit 130 displays the first final symptom indexes or the second final symptom indexes for each pollen generation species on the graph of the allergy generation risk grades for each symptom index. Thereafter, the allergy generation risk grade determination unit 130 extracts the allergy generation risk grades for each pollen generation species according to the area in which the first final symptom indexes or the second final symptom indexes for each pollen generation species are located on the graph of the allergy generation risk grades for each symptom index.

In another embodiment, the allergy generation risk grade determination unit 130 extracts the date corresponding to the same pollen generation species and the same pollen generation grade in the pollen calendar. For example, suppose a pollen generation grade for oak species is maintained at “high” from April 20 to April 30 in the pollen calendar. In this case, the allergy generation risk grade determination unit 130 extracts the date from April 20 to April 30 in the pollen calendar. Then, the allergy generation risk grade determination unit 130 extracts the first final symptom index and/or the second final symptom index recorded on the date corresponding to the date extracted from the pollen calendar among the dates of the personal allergic symptom diary. The extracted first final symptom index and/or second final symptom index is displayed on the graph of the allergy generation risk grades for each index. Then, the allergy generation risk grade determination unit 130 extracts the allergy generation risk grades for the oak species according to the area in which the first final symptom index or the second final symptom index is located on the graph of the allergy generation risks for each symptom index. The allergy generation risk grade determination unit 130 repeats this process to extract the allergy generation risk grades for each pollen generation species.

In the above embodiment, the allergy generation risk grade determination unit 130 determines the higher of the two allergy generation risk grades as the final allergy generation risk grade of the user when the difference between the allergy generation risk grade corresponding to the first final symptom index and the allergy generation risk grade corresponding to the second final symptom index is greater than or equal to the reference value.

In conclusion, the allergy generation risk grade determination unit 130 may determine the pollen generation species whose final allergy generation risk grade is higher than or equal to a specific grade among the final allergy generation risk grades extracted for each pollen generation species as the allergy-sensitive tree species of the user.

The personalized pollen calendar generation unit 140 stores the allergy generation risk grade determined by the allergy generation risk grade determination unit 130 to generate the personalized pollen calendar.

For example, the personalized pollen calendar generation unit 140 may compare the time when the allergy generation risk grades for each pollen generation species of each user persist to the specific grade or higher and the time when the pollen generation grades extracted from the pollen calendar persist to the specific grade or higher to determine the allergy-sensitive tree species for each user and then generate the personalized pollen calendar in which the allergic symptoms are displayed.

As another example, the personalized pollen calendar generation unit 140 may compare the time when the daily symptom indexes of the user are maintained at a certain level or more and the time when the allergy generation risk grades for each pollen generation species are maintained at a specific level to determine the personal allergy-sensitive tree species, thereby generating the personalized pollen calendars in which the allergic symptoms are displayed.

The risk forecast generation unit 150 applies the allergy generation risk grades for each pollen generation species and the allergy-sensitive tree species to the Korea Metrological Administration pollen forecast to generate the personalized risk forecast for each city and county in which the information on the region in which the allergic symptoms may appear is spatially represented in more detail compared to the Korea Metrological Administration pollen forecast.

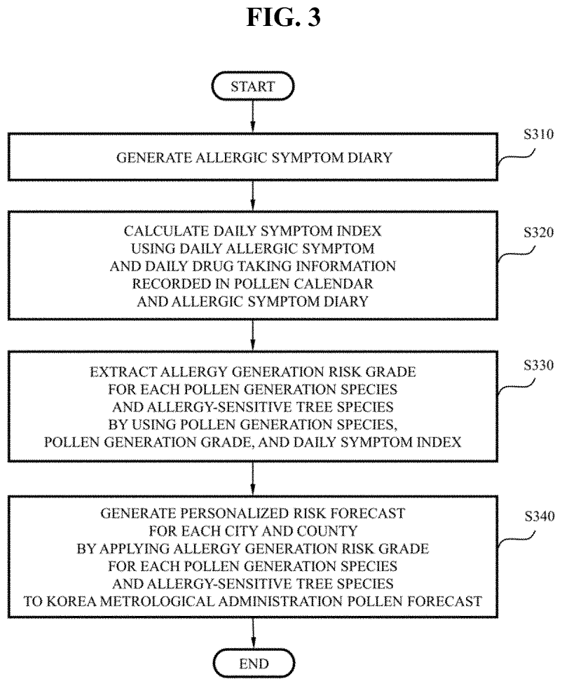

Hereinafter, a method of predicting a personalized pollen allergy according to an embodiment of the present disclosure will be described with reference to FIGS. 3 to 5.

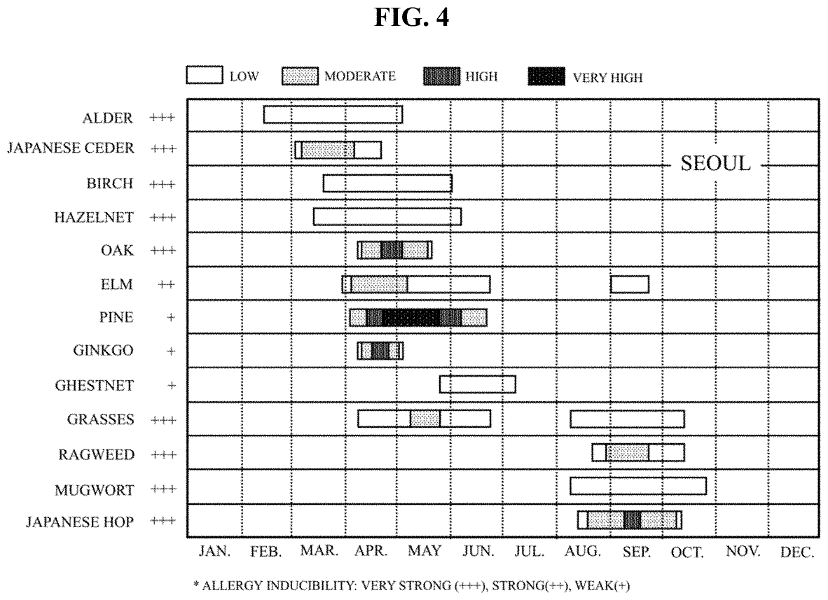

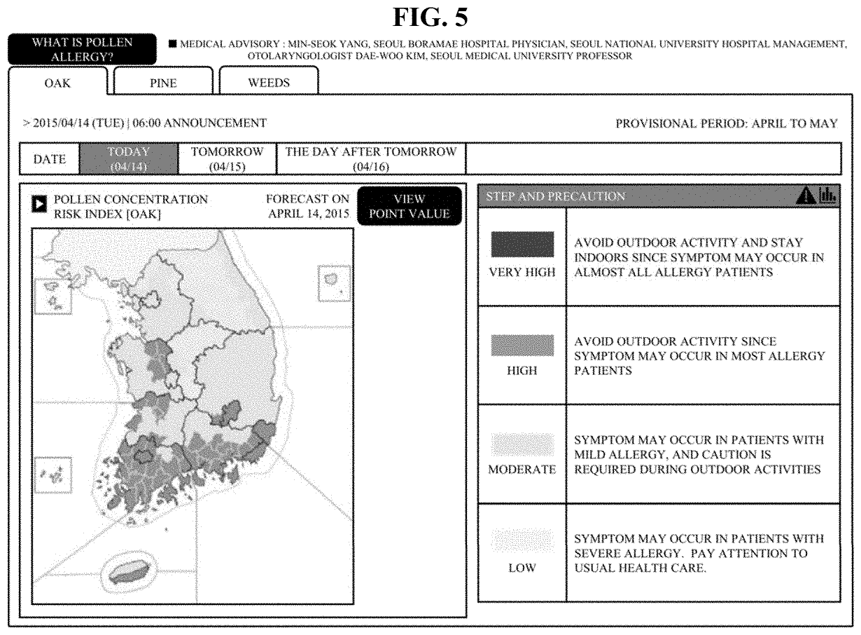

FIG. 3 is a flowchart for describing a method of predicting a personalized pollen allergy according to an embodiment of the present disclosure. FIGS. 4 and 5 are exemplary views for explaining an execution process of FIG. 3. Specifically, FIG. 4 is a diagram illustrating a pollen calendar in Seoul, Korea. FIG. 5 is a diagram illustrating a Korea Metrological Administration pollen forecast screen provided by the server illustrated in FIG. 2.

Referring to FIG. 3, the server 100 generates the personal allergic symptom diary by recording the daily allergic symptom and daily drug taking information provided from the user terminals 200_1 to 200_N (operation S310).

Thereafter, the server 100 calculates the daily symptom indexes using the daily allergic symptom and daily drug taking information that are recorded in allergy patient symptom diary and the pollen calendar of the region corresponding to the location of the user (operation S320).

In one embodiment for operation S320, the server 100 may calculate the symptom indexes for each pollen generation species by region (by city) by applying the daily symptom indexes to the pollen calendar by region (by city).

The server 100 extracts the allergy generation risk grades for each pollen generation species and the allergy-sensitive grade of the user by using the pollen generation species and the pollen generation grades extracted on the pollen calendar, and the calculated daily symptom indexes (operation S330).

In one embodiment for operation S330, the server 100 may compare the time when the allergy generation risk grades for each pollen generation species persist to the specific grade or higher and the time when the pollen generation grades extracted from the pollen calendar persist to the specific grade or higher to determine the allergy-sensitive tree species of the user and then generate the personalized pollen calendar in which the allergic symptoms are displayed.

For example, when the pollen generation grade of oak species is “high” or more, the time when the allergy inducibility is predicted to be “very strong” may be predicted from April 20 to May 2 in the case of Seoul (see FIG. 4). Although not illustrated in the drawing, the time when the allergy inducibility is predicted to be “very strong” may be predicted to be April 10 to May 5 in the case of Daejeon, April 20 to April 30 in the case of Daegu, April 14 to May 5 in the case of Jeonju, and April 18 to May 4 in the case of Gwangju. In addition, it may be predicted that there is no risk for Gangneung, Busan, and Jeju.

The server 100 applies the allergy generation risk grade for each pollen generation species and the allergy-sensitive tree species to the Korea Metrological Administration pollen forecast to generate a personalized risk forecast for each city and county in which information on a region in which allergic symptoms may appear is spatially represented in more detail compared to the Korea Metrological Administration pollen forecast (operation S340).

Accordingly, the user may adjust an outdoor activity region and an outdoor activity time with reference to such personalized information, or take action in advance using drugs or the like.

According to the present disclosure, the Korea Metrological Administration pollen forecast may be provided as a three-day forecast for each city and county for oak, pine, and weeds, as illustrated in FIG. 5. In this forecast service, weeds are developed with Japanese hop pollen as a representative tree species. Therefore, the server 100 may apply the personalized risk information to the Korea Metrological Administration pollen forecast when the allergy-sensitive tree species of the user is oak, pine, or Japanese hop to produce a three-day forecast for each city and county that is spatially represented in more detail than the previously produced forecast information by city.

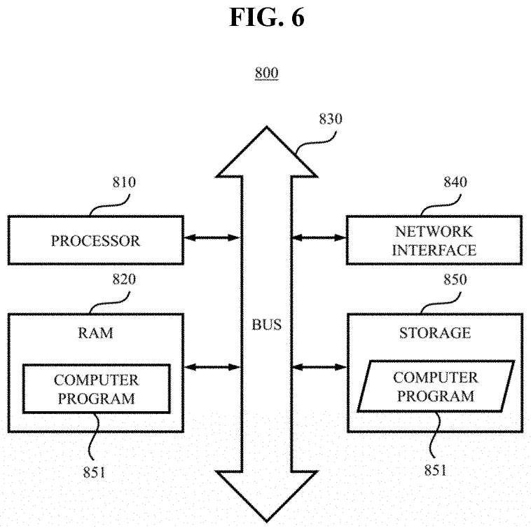

Hereinabove, the method of predicting a personalized pollen allergy according to the embodiment of the present disclosure and the server 100 performing the same have been described with reference to FIGS. 1 to 5. Hereinafter, an exemplary computing device capable of implementing the server 100 according to some embodiments of the present disclosure will be described with reference to FIG. 6.

Referring to FIG. 6, a computing device 800 may include one or more processors 810, a storage 850 for storing a computer program 851, a memory 820 for loading a computer program 851 run by the processor 810, a bus 830, and a network interface 840. However, only the components related to the embodiment of the present disclosure are illustrated in FIG. 6. Accordingly, those skilled in the art to which the present disclosure pertains may understand that other general-purpose components other than those illustrated in FIG. 6 may be further included.

The processor 810 controls an overall operation of each component of the computing device 800. The processor 810 may be configured to include a central processing unit (CPU), a micro processor unit (MPU), a micro controller unit (MCU), a graphic processing unit (GPU), or any type of processor well known in the art of the present disclosure. In addition, the processor 810 may perform an operation on at least one computer program for performing the method of predicting a personalized pollen allergy according to embodiments of the present disclosure. The computing device 800 may include one or more processors.

The memory 820 stores data for supporting various functions of the computing device 800. The memory 820 stores a plurality of computer programs (app, application program, or application software) run in the computing device 800, and data, instructions, and one or more pieces of information for the operation of the computing device 800. At least some of the computer programs may be downloaded from an external device (not illustrated). In addition, at least some of the computer programs may be installed in the computing device 800 from the time of shipment for basic functions (e.g., receiving a message and sending a message) of the computing device 800.

Meanwhile, the memory 820 may load one or more computer programs 851 from the storage 850 to perform the method of predicting a personalized pollen allergy according to the embodiments of the present disclosure. In FIG. 6, a random access memory (RAM) is illustrated as an example of the memory 820.

The bus 830 provides a communication function between the components of the computing device 800. The bus 830 may be implemented as various types of buses such as an address bus, a data bus, and a control bus.

The network interface 840 supports wired/wireless Internet communication of the computing device 800. In addition, the network interface 840 may support various communication methods other than the Internet communication. To this end, the network interface 840 may include a communication module well known in the art of the present disclosure.

The storage 850 may non-transitorily store one or more computer programs 851. The storage 850 may be configured to include a nonvolatile memory, such as a read only memory (ROM), an erasable programmable ROM (EPROM), an electrically erasable programmable ROM (EEPROM), and a flash memory, a hard disk, a removable disk, or any well-known computer-readable recording medium in the art to which the present disclosure belongs.

Hereinabove, an exemplary computing device capable of implementing the server 100 according to some embodiments of the present disclosure will be described with reference to FIG. 6. The computing device illustrated in FIG. 6 may not only implement the server 100 according to some embodiments of the present disclosure but may also implement the user terminals 200_1 to 200_N according to some embodiments of the present disclosure. In this case, the computing device 800 may further include an input unit and an output unit in addition to the components illustrated in FIG. 6.

The input unit may include a camera for receiving a video signal, a microphone for receiving an audio signal, and a user input unit for receiving information from a user. The user input unit may include one or more of a touch key and a mechanical key. Video data collected through the camera or the audio signal collected through the microphone may be analyzed and may be processed as control commands of the user.

The output unit is for visually, auditorily, or tactilely outputting the command processing result, and may include a display unit, an optical output unit, a speaker, a haptic output unit, and an optical output unit.

Meanwhile, components constituting the server 100 or the user terminals 200_1 to 200_N may be implemented as modules.

The module refers to software or hardware components such as a field programmable gate array (FPGA) or an application specific integrated circuit (ASIC), and the module performs certain roles. However, the module is not meant to be limited to software or hardware. The module may be stored in a storage medium that may be addressed or may be configured to run one or more processors. Accordingly, for example, the “module” includes components such as software components, object-oriented software components, class components, and task components, and includes processors, functions, attributes, procedures, subroutines, segments of a program code, drivers, firmware, a microcode, a circuit, data, a database, data structures, tables, arrays, and variables. The functions provided by the components and modules may be combined into a smaller number of components and modules or further divided into additional components and modules.

According to the present disclosure, it is possible to provide a personalized pollen allergy prediction service by combining a pollen calendar with a patient's pollen allergic symptoms.

Although described with reference to the limited embodiments and drawings, the present disclosure is not limited to the above embodiments. It is obvious to those of ordinary skill in the art to which the present disclosure pertains that other modifications based on the technical idea of the present disclosure may be implemented in addition to the embodiments disclosed herein. Therefore, the scope and spirit of the present disclosure should be understood only by the following claims, and all of the equivalences and equivalent modifications to the claims are intended to fall within the scope and spirit of the present disclosure.

What is claimed is:

1. A method of predicting a personalized pollen allergy performed in a personalized pollen allergy prediction server, the method comprising:
generating a personal allergic symptom diary by recording a daily allergic symptom and daily drug taking information of a user;
calculating a daily symptom index using a pollen calendar of a region corresponding to a location of the user and the daily allergic symptom;
extracting allergy generation risk grades for each pollen generation species and allergy-sensitive tree species of the user by using the pollen generation species and a pollen generation grade extracted from the pollen calendar, and the daily symptom index, and generating a personalized pollen calendar based on the extracted information; and
generating a personalized risk forecast for each city and county for the user by applying the allergy generation risk grades for each pollen generation species and the allergy-sensitive tree species to a Metrological Administration pollen forecast.

2. The method of claim 1, wherein the calculating of the daily symptom index includes calculating symptom indexes for each pollen generation species by applying the daily symptom index to pollen calendars by region.

3. The method of claim 1, wherein the generating of the personalized pollen calendar includes generating the personalized pollen calendar by comparing a time when the allergy generation risk grades for each pollen generation species persist to a specific grade or higher and a time when the pollen generation grade extracted from the pollen calendar persists to the specific grade or higher to determine the allergy-sensitive tree species and then display an allergic symptom.

4. The method of claim 1, wherein the generating of the daily symptom index includes calculating the daily symptom index by counting the daily allergic symptom in different ways depending on whether the user is taking a drug,
the daily allergic symptom includes one or more of a primary symptom and a secondary symptom,
the primary symptom includes one or more of sneezing, clear nasal discharge, stuffy nose, nasal itching, and difficulty in smelling, and
the secondary symptom includes one or more of headache, mouth breathing, post nasal drip syndrome, coughing while sleeping, and sleep disorder.

5. A personalized pollen allergy prediction server comprising:
an allergic symptom diary generation unit configured to generate a personal allergic symptom diary by recording a daily allergic symptom and daily drug taking information of a user;
a symptom index calculation unit configured to calculate a daily symptom index using a pollen calendar of a region corresponding to a location of the user and the daily allergic symptom;
a personalized pollen calendar generation unit configured to extract allergy generation risk grades for each pollen generation species and allergy-sensitive tree species of the user by using the pollen generation species and a pollen generation grade extracted from the pollen calendar, and the daily symptom index, and generate a personalized pollen calendar based on the extracted information; and
a risk forecast generation unit configured to generate a personalized risk forecast for each city and county for the user by applying the allergy generation risk grades for each pollen generation species and the allergy-sensitive tree species to a Metrological Administration pollen forecast.

6. The personalized pollen allergy prediction server of claim 5, wherein the symptom index calculation unit calculates symptom indexes for each pollen generation species by applying the daily symptom index to pollen calendars by region.

7. The personalized pollen allergy prediction server of claim 5, wherein the personalized pollen calendar generation unit generates the personalized pollen calendar by comparing a time when the allergy generation risk grades for each pollen generation species persist to a specific grade or higher and a time when the pollen generation grade extracted from the pollen calendar persists to the specific grade or higher to determine the allergy-sensitive tree species and then display an allergic symptom.

8. The personalized pollen allergy prediction server of claim 5, wherein the symptom index calculation unit calculates the daily symptom index by counting the daily allergic symptom in different ways depending on whether the user is taking a drug,
the daily allergic symptom includes one or more of a primary symptom and a secondary symptom,
the primary symptom includes one or more of sneezing, clear nasal discharge, stuffy nose, nasal itching, and difficulty in smelling, and
the secondary symptom includes one or more of headache, mouth breathing, post nasal drip syndrome, coughing while sleeping, and sleep disorder.
